(19) United States
(12) Patent Application Publication
(10) Pub. No.: US 2007/0110833 A1
(43) Pub. Date: May 17, 2007

(54) Composition comprising the alcohol compound isolated from the extract of cucurbitaceae family plant having anti-adipogenic and anti-obesity activity

(76) Inventors: Mi Rim Jin, Seoul (KR); Jae Ha Ryu, Seoul (KR); Hyoun Jeong Choi, Incheon (KR); Hyun Jin Jung, Seoul (KR); Kyoung Chul Park, Seoul (KR); Sun Young Kim, Seoul (KR)

Correspondence Address: KIRK HAHN, 14431 HOLT AVE, SANTA ANA, CA, 92705, US

(21) Appl. No.: 10/581,575

(22) PCT Filed: Dec. 3, 2004
(86) PCT No.: PCT/KR04/03168

§ 371 Date: Jun. 2, 2006

(30) Foreign Application Priority Data
Dec. 3, 2003 (KR) 10-2003-0087280

(51) Int. Cl. A61K 36/42

(52) U.S. Cl. 424/758

(57) ABSTRACT

The present invention is related to an extract of Cucurbitaceae family plant or the purified extract isolated therefrom having anti-adipogenic and anti-obesity activity, and a composition comprising the same. The extract of Cucurbitaceae family plant of the present invention showed potent reducing activity of body weight, decreasing effect on the blood triglyceride and cholesterol level, activating activity of PPAR α and δ, reducing activity of the gene expression of stearoyl-CoA desaturase, and preventing activity from the adipogenesis of precursor fat cells with no toxicity, therefore, those extract can be useful in treating or preventing obesity and adipogenesis-involved diseases as a medicine or health care food.

### TECHNICAL FIELD

The present invention relates to a composition comprising the extract of Cucurbitaceae family plant or the purified extract isolated therefrom having anti-adipogenic and anti-obesity activity.

### BACKGROUND ART

Adipogenesis is a process to differentiate preadipocytes into mature adipocytes and accumulate lipids in cytoplasmic organells named of lipid droplets, which is known to be a risky factor which may give rise to various adult diseases such as obesity, diabetes, steatosis and coronary heart disease. Precursor fat cells such as fibroblasts can be differentiated into mature ones resulting in the formation of lipid droplets within them. The differentiation mechanism has been studied by using specific cell lines such as 3T3-L1. Adipocyte differentiation is a complex process accompanied by coordinated changes in morphology, hormone sensitivity, and gene expression. These changes are regulated by several transcription factors such as C/EBPs (CAAT enhancer binding proteins), PPARs (Peroxisome Proliferator-Activated receptors), and ADD/SREBPs (Adipocyte determination and differentiation dependent factor 1/sterol regulatory element-binding proteins). (Bart A Jessen et al., Gene, 299, pp95-100, 2002; Darington et al., J. Biol. Chem., 23 pp30057-30060, 1998; Brun R. P et al., Curr. Opin Cell. Biol., 8, pp826-832, 1996). These transcription factors are induced at different stages of adipocyte differentiation and functionally interact with each other to conduct adipogenesis and lipogenesis by regulating gene expression. For example, C/EBP beta and delta factors are temporally overexpressed by the external hormonal stimuli such as MDI (isobutylmethylxanthin, dexamethason and insulin), which triggers adipocyte differentiation process. (Reusch J. E. et al., Mol. Cell. Biol., 20, pp1008-1020, 2000). Subsequently, they induce the increase of C/EBP alpha and PPAR gamma (James M. N. et al., J. Nutr., 130, pp3122S-3126S, 2000). Especially, PPAR gamma is predominantly expressed in adipocytes and is a key determination transcription factor for adipogenesis, which forms a heterodimer with RXR (Retinoic acid X receptor) and binds to PPRE (Peroxisome Proliferator Response elements) found in promoters of various genes involved in adipogenesis (Tontonoz P. E. et al., Genes Dev., 8, pp1224-1234, 1994). The interaction between C/EBP alpha and PPAR gamma is crucial in the adipocyte differentiation and those factors control the expression of adipocyte-specific genes such as fatty aid bound protein, aP2 and lipid metabolic enzymes. Especially, ADD1/SREBPs also plays a key role for lipogenesis and insulin-stimulated gene expression, and the expression of ADD 1/SREBP1c contributes to the activation of PPAR gamma (Rosen E. D. et al., Annu. Rev. Cell Dev. Biol., 16, pp145-171, 2000; Osborn T. F., J. Biol. Chem., X, pp32379-32382, 2000). The adipocytes finished the differentiation process synthesize lipids and store them in a form of triglycerides.

In the meanwhile, the homeostasis of lipid metabolism is maintained by the balance between synthesis and disintegration of fat. ADD1/SREBP1 controls the synthesis of fatty acid, triglyceride, cholesterol, and phospholipid etc (Horton J. D. et al., J. Clin. Invest., 109, pp1125-1131, 2002). SREBPs are synthesized as about 1150 amino acid precursors bound to the endoplasmic reticulum and nuclear envelope. To be active, the membrane-bound SREBP must be proteloytically cleaved to release the N-terminal segment so that it can enter the nucleus. The cleaved SREBPs, designated the nuclear form, binds to the SRE (sterol regulated elements) in the regulatory gene promoter. The genes regulated by SREBP1c, one of the SREBP isoforms are ACL (ATP citrate lyase), ACC (Acetyl CoA Carboxylase), FAS (Fatty acid synthase), and SCD (Stearoly-CoA desarurase) etc (Osborn T. F. et al., J. Biol. Chem., 275, pp32379-32382, 2000; Soazig L. L. et al., J. Biol. Chem., 277, pp35625-35634, 2002). It has been reported that PPAR alpha plays an important role in regulating lipolysis (Beisiegel U., Proc. Natl. Acad. Sci U.S.A., 26, pp13656-13661, 1999) through control of lipid metabolic enzymes such as LPL (lipoprotein lipase), apoproteins, ACO (Acyl-CoA oxidase) and thiolase (Dreyer C et al., Cell, 68, pp879-887, 1992).

Obesity results from a chronic imbalance between energy intake and energy expenditure, resulting in increased fat storage. The mechanism of obesity is not fully understood however, the complex interactions of neural, hormonal, genetic and environmental factors due to Westernized diet are thought to induce this obesity epidemic. Over accumulation of fat might be a high risk factor for various metabolic syndromes such as diabetes, hypertension, dyslipidaemia and cardiovascular disease. (Manson et al., New England J. Med., 333 pp677-685, 1995; Kopleman P. G., Nature, 404 pp635-643, 2000; Must et al., JAMA 282, pp1523-1529. 1999).

Although there are several well-known representative anti-obesity agents such as Xenical™ (Roche Pharm. Co Ltd. Swiss), Reductil™ (Abbot Co Ltd. USA) and Exolise™ (Atopharma Co Ltd. France), more effective agents have been needed because of their side effects such as heart disease, respiratory disease and neuronal system disorder.

Recent strategies for developing anti-obesity agent are focused on reducing diet, inhibiting calorie intake, stimulating thermogenic reaction, regulating energy metabolism, and controlling signal transduction through neuronal nerve system (Kopleman P. G., Nature, 404 pp635-643, 2000). There have been many attempts to develop effective anti-obesity agents, however satisfactory drugs showing potent efficacy as well as safety have not been developed yet.

Accordingly, the attempts to develop an effective anti-obesity agent with natural products of which safety has been verified are more effective rather than them with synthetic substance.

Most of the plants belonged to Cucurbitaceae family of Dicotyledonaceae class are annual or perrenial viny plants and distributed in tropical and subtropical zone. Among them, Melothris japonica, Schzopepon bryoniaefolius, Gynostemma pentaphyllum and the like have been distributed, and pumpkin (Cucurbita moschata DUCH), water melon (Citrullus vulgaris SCHRAD), sponge gourd (Luffa cylindrical L. ROEM), cucumber (Cucumis sativus L) and the like have been activated in Korea.

It has been reported that pumpkin (Cucurbita moschata DUCH) comprising cucurbitine and fat oils such as linoleic acid, oleic acid, carotene etc shows anthelmintic activity; water melon (Citrullus vulgaris SCHRAD) comprising citrulline, alanine, fructose, glucose etc shows potent diuretic activity; sponge gourd (Luffa cylindrical L. ROEM) is used as a washing tool; and cucumber (Cucumis sativus L) comprising glycoside, caffeic acid, cucurbitacins etc shows diuretic activity according to the literature (Chung B. S et al: HyangyakDaesajeon, young-rim press, pp 945-957, 1998).

However, there has been not reported or disclosed on the preventing or treating activity of the extract of plant belonged to Cucurbitaceae family showing potent anti-obesity effect in any of above cited literatures, the disclosures of which are incorporated herein by reference.

To investigate the anti-obesity effect of the extract of plant belonged to Cucurbitaceae family, the inventors of present invention have intensively carried out various in vitro experiment concerning with the inhibition effect on the differentiation of adipocyte and triglycerides, the expression of gene correlated to adipocyte differentiation such as PPAR gamma factor, ACO I, Apo CIII etc, and animal test concerning with the inhibition effect on the accumulation of adipocyte and triglycerides, and the reducing effect on the body weight of test animals. As a result of the investigation, the inventors finally completed the present invention by confirming that the plant extract of the present invention strongly inhibited the accumulation of adipocyte and triglycerides, and reduced the body weight and it can be useful as a potent anti-obesity agent.

### DISCLOSURE

Accordingly, the present invention provides a pharmaceutical composition comprising the extract of Cucurbitaceae family plant or the purified extract isolated therefrom having anti-adipogenic and anti-obesity activity as an active ingredient in an effective amount to treat and prevent obesity and adipogenesis-involved diseases.

The term ‘the purified extract’ disclosed herein comprises inventive ‘CMC-9’ designated by the present inventors, which could be prepared by the steps consisting of: adding about 5 to 15 fold volume of distilled water to dried material of Cucurbitaceae family plant, extracting the plant to obtain plant extract, filtrating and drying the extract with reduced pressure to obtain hot water-soluble extract of the plant at 1^{st} step; suspending said hot water-soluble extract with water and subjecting fractionation with hexane, chloroform, ethylacetate, butanol solvent with increasing order of polarity to obtain respective organic solvent-soluble fraction at 2^{nd} step; subjecting said chloroform-soluble fraction to silica gel column chromatography with a solvent mixture mixed with hexane: chloroform: methanol (16:15:1) to afford 11 sub-fractions at 3^{rd} step; subjecting 9^{th} fraction among said sub-fractions to repetitive silica gel column chromatography with a solvent mixture mixed with chloroform: methanol and HPLC to obtain inventive purified extract designated to cmc-‘9’ showing TLC spectrum as can be seen in FIG. 12.

Therefore, the present invention provides with novel cmc-9 purified extract prepared by above described method, which has potent anti-adipogenecity and anti-obesity activity and the process for preparing the same.

The term ‘the Cucurbitaceae family plant’ disclosed herein comprises pumpkin (Cucurbita moschata DUCH), water-melon (Citrullus vulgaris SCHRAD), sponge gourd (Luffa cylindrical L. ROEM), gourd (Lagenaria siceraria STANDL. var. depressa HERA), and cucumber (Cucumis sativus L), preferably, pumpkin (Cucurbita moschata DUCH), water melon (Citrullus vulgaris SCHRAD), sponge gourd (Luffa cylindrical L. ROEM).

Above-described ‘material’ comprises herb, fruit, stem and leaf, preferably, the stem or leaf of Cucurbitaceae family plant.

Above-described ‘extract’ comprises crude extract or non-polar solvent soluble extract of the herb, fruit, stem and leaf, preferably, the stem or leaf of Cucurbitaceae family Cucurbitaceae family plant.

Accordingly, It is an object of the present invention to provide a pharmaceutical composition comprising the crude extract or non-polar solvent soluble extract of Cucurbitaceae family plant, as an active ingredients for the treatment and prevention of obesity and adipogenesis-related diseases disease.

The term ‘crude extract’ disclosed herein comprises the extract prepared by extracting plant material with water, lower alcohols such as methanol, ethanol, preferably methanol and the like, or the mixtures thereof.

The term ‘non-polar solvent soluble extract’ disclosed herein can be prepared by extracting above crude extract with non-polar solvent, for example, hexane, ethyl acetate or dichloromethane, preferably ethyl acetate.

It is an object of the present invention to provide a use of a crude extract, non-polar solvent soluble extract or above-described cmc-9 extract isolated from Cucurbitaceae family plant for the preparation of therapeutic agent for the treatment and prevention of obesity and adipogenesis-involved diseases in a mammal including human in need thereof.

It is an object of the present invention to provide a method of treating or preventing obesity and adipogenesis-involved diseases in a mammal comprising administering to said mammal an effective amount of a crude extract, non-polar solvent soluble extract or above-described cmc-9 extract isolated from Cucurbitaceae family plant, together with a pharmaceutically acceptable carrier thereof.

It is another object of the present invention to provide a health care food or food additives comprising a crude extract, non-polar solvent soluble extract or above-described cmc-9 extract isolated from Cucurbitaceae family plant, together with a sitologically acceptable additive for the prevention and alleviation of obesity and adipogenesis-involved diseases.

The term ‘obesity and adipogenesis-involved diseases’ disclosed herein comprises obesity, type II diabetes, steatosis, hyperlipemia, cardiovascular disease, artherosclerosis and the like.

The pharmaceutical composition of the present invention can contain about 0.02-90% by weight of the above extract based on the total weight of the composition.

The health care food of the present invention comprises the above extract as 0.01 to 80%, preferably 1 to 50% by weight based on the total weight of the composition.

Above health care food can be contained in health care food, health beverage etc, and may be used as powder, granule, tablet, chewing tablet, capsule, beverage etc.

An inventive crude extract or non-polar solvent soluble extract of Cucurbitaceae family plant may be prepared in accordance with the following preferred embodiment.

Hereinafter, the present invention is described in detail.

An inventive crude extract or non-polar solvent soluble extract of Cucurbitaceae family plant can be prepared in detail by following procedures,

The inventive crude extract of Cucurbitaceae family plant can be prepared by follows; the stem or leaf of Cucurbitaceae family plant such as pumpkin, water melon or sponge gourd is dried, cut, crushed and mixed with 1 to 25-fold, preferably, approximately 5 to 15 fold volume of distilled water, lower alcohols such as methanol, ethanol, butanol and the like, or the mixtures thereof, preferably methanol; the solution is treated with hot water at the temperature ranging from 20 to 100° C., preferably from 70 to 100° C., for the period ranging from 30 min to 24 hours, preferably, 30 min to 3 hours with extraction method such as extracting with hot water, acid water, reflux extraction, or ultra-sonication extraction with 1 to 5 times, preferably 2 to 3 times, consecutively; the residue is filtered to obtain the supernatant to be concentrated with rotary evaporator, at the temperature ranging from 20 to 100° C., preferably from 50 to 70° C. and then dried by vacuum freeze-drying, hot air-drying or spray drying to obtain dried crude extract powder of crude extract of Cucurbitaceae family plant which can be soluble in water, lower alcohols, or the mixtures thereof.

Additionally, polar-solvent soluble and non-polar solvent soluble extract of present invention can be prepared by following procedure; the crude extract prepared by above described step, is suspended in water, and then is mixed with 1 to 100-fold, preferably, 1 to 5-fold volume of non polar solvent such as ethyl acetate, chloroform, hexane and the like; the non-polar solvent soluble layer is collected to obtain non-polar solvent soluble extract of the present invention and remaining polar solvent soluble layer is collected to obtain polar solvent soluble extract of the present invention which is soluble in water, lower alcohols, or the mixtures thereof. Also, above described procedures may be modified or subjected to further step to fractionate or isolate more potent fractions or compounds by conventional procedure well-known in the art, for example, the procedure disclosed in the literature (Harborne J. B. Phytochemical methods: A guide to modern techniques of plant analysis, 3^{rd} Ed. pp6-7, 1998).

Additionally, inventive ‘CMC-9’ designated by the present inventors, which could be prepared by subjecting chloroform soluble fraction showing most potent anti-adipogenic and anti-obesity activity to silica gel column chromatography with a solvent mixture mixed with hexane: chloroform:methanol (16:15:1) to afford 11 sub-fractions; subjecting 9 faction among said sub-fractions showing most potent anti-adipogenenic and anti-obesity activity to repetitive silica gel column chromatography with a solvent mixture mixed with chloroform:methanol (30:1) and HPLC using methanol ranging from 20 to 70% as a mobile phase and running 40% methanol with a flow velocity of 2 ml/m to obtain inventive ‘cmc-9’ extract eluting at 26.8 min which shows 0.32 of R value in TLC eluting solvent system (chloroform:methanol=20:1) as can be seen in FIG. 12.

To investigate the effect of inventive extract on the anti-obesity effect, the inventors of present invention have intensively carried out various in vitro experiment concerning with the inhibition effect on the differentiation of adipocyte and triglycerides, the expression of gene correlated to adipocyte differentiation such as PPAR gamma factor, ACO I, Apo CIII etc, and animal test concerning with the inhibition effect on the accumulation of adipocyte and triglycerides, and the reducing effect on the body weight of test animals. As a result of the investigation, the inventors confirmed that the plant extract of the present invention strongly inhibited the accumulation of adipocyte and triglycerides, and reduced the body weight therefore it can be useful as a potent anti-obesity agent.

The extract according to the present invention can be provided as a pharmaceutical composition containing pharmaceutically acceptable carriers, adjuvants or diluents.

For example, the extract of the present invention can be dissolved in oils, propylene glycol or other solvents which are commonly used to produce an injection. Suitable examples of the carriers include physiological saline, polyethylene glycol, ethanol, vegetable oils, isopropyl myristate, etc., but are not limited to them. For topical administration, the extract of the present invention can be formulated in the form of ointments and creams.

The extract of the present invention has potent anti-obesity and anti-adipogenic activity, and the pharmaceutical composition of the present invention thus may be employed to treat or prevent obesity and adipogenesis-related diseases.

Hereinafter, the following formulation methods and excipients are merely exemplary and in no way limit the invention.

The extract of the present invention in pharmaceutical dosage forms may be used in the form of their pharmaceutically acceptable salts, and also may be used alone or in appropriate association, as well as in combination with other pharmaceutically active compounds.

The extract of the present invention may be formulated into preparations for injections by dissolving, suspending, or emulsifying them in aqueous solvents such as normal saline, 5% Dextrose, or non-aqueous solvent such as vegetable oil, synthetic aliphatic acid glycerides, esters of higher aliphatic acids or propylene glycol. The formulation may include conventional additives such as solubilizers, isotonic agents, suspending agents, emulsifying agents, stabilizers and preservatives.

The desirable dose of the inventive extract varies depending on the condition and the weight of the subject, severity, drug form, route and period of administration, and may be chosen by those skilled in the art. However, in order to obtain desirable effects, it is generally recommended to administer at the amount ranging 0.0001-100 mg/kg, preferably 0.001-100 mg/kg by weight/day of the inventive extract of the present invention. The dose may be administered in single or divided into several times per day. In terms of composition, the extract should be present between 0.0001 to 10% by weight, preferably 0.0001 to 1% by weight based on the total weight of the composition.

The pharmaceutical composition of present invention can be administered to a subject animal such as mammals (rat, mouse, domestic animals or human) via various routes. All modes of administration are contemplated, for example, administration can be made orally, rectally or by intravenous, intramuscular, subcutaneous, intrathecal, epidural or intracerebroventricular injection.

The extract of the present invention also can be used as a main component or additive and aiding agent in the preparation of various functional health food and health care food.

The term ‘a functional health food’ defined herein means ‘the functional food having enhanced functionality such as physical functionality or physiological functionality by adding the compound of the present invention to conventional food to prevent or improve obesity and adipogenesis-involved diseases in human or mammal.

It is the other object of the present invention to provide a health care food comprising a crude extract, non-polar solvent soluble extract or above-described cmc-9 extract isolated from Cucurbitaceae family plant, together with a sitologically acceptable additive for the prevention and alleviation of obesity and adipogenesis-involved disease.

The term ‘a health care food’ defined herein means ‘the food containing the extract of the present invention showing no specific intended effect but general intended effect in a small amount of quantity as a form of additive or in a whole amount of quantity as a form of capsule, pill, tablet etc.

The term ‘a sitologically acceptable additive’ defined herein means ‘any substance the intended use which results or may reasonably be expected to result-directly or indirectly-in its becoming a component or otherwise affecting the characteristics of any food’, for example, thickening agent, maturing agent, bleaching agent, sequesterants, humectant, anticaking agent, clarifying agents, airing agent, emulsifier, stabilizer, thickner, bases and acid, foaming agents, nutrients, coloring agent, flavoring agent, sweetner, preservative agent, antioxidant, etc, which shall be explained in detail as follows.

If a substance is added to a food for a specific purpose in that food, it is referred to as a direct additive and indirect food additives are those that become part of the food in trace amounts due to its packaging, storage or other handling.

Above described health foods can be contained in food, health beverage, dietary therapy etc, and may be used as a form of powder, granule, tablet, chewing tablet, capsule, beverage etc for preventing or improving obesity and adipogenesis-involved diseases.

Also, above described extract can be added to food or beverage for prevention and improvement of obesity and adipogenesis-involved diseases. The amount of above described extract in food or beverage as a functional health food or health care food may generally range from about 0.01 to 100 w/w % of total weight of food for functional health food composition. In particular, although the preferable amount of the extract of the present invention in the functional health food, health care food or special nutrient food may be varied in accordance to the intended purpose of each food, it is preferably used in general to use as a additive in the amount of the extract of the present invention ranging from about 0.01 to 5% in food such as noodles and the like, from 40 to 100% in health care food on the ratio of 100% of the food composition.

Providing that the health beverage composition of present invention contains above described extract as an essential component in the indicated ratio, there is no particular limitation on the other liquid component, wherein the other component can be various deodorant or natural carbohydrate etc such as conventional beverage. Examples of aforementioned natural carbohydrate are monosacharide such as glucose, fructose etc; disacharide such as maltose, sucrose etc; conventional sugar such as dextrin, cyclodextrin; and sugar alcohol such as xylitol, and erythritol etc. As the other deodorant than aforementioned ones, natural deodorant such as taurnatin, stevia extract such as levaudioside A, glycyrrhizin et al., and synthetic deodorant such as saccharin, aspartam et al., may be useful favorably. The amount of above described natural carbohydrate is generally ranges from about 1 to 20 g, preferably 5 to 12 g in the ratio of 100 ml of present beverage composition.

The other components than aforementioned composition are various nutrients, a vitamin, a mineral or an electrolyte, synthetic flavoring agent, a coloring agent and improving agent in case of cheese, chocolate et al., pectic acid and the salt thereof, alginic acid and the salt thereof, organic acid, protective colloidal adhesive, pH controlling agent, stabilizer, a preservative, glycerin, alcohol, carbonizing agent used in carbonate beverage et al. The other component than aforementioned ones may be fruit juice for preparing natural fruit juice, fruit juice beverage and vegetable beverage, wherein the component can be used independently or in combination. The ratio of the components is not so important but is generally range from about 0 to 20 w/w % per 100 w/w % present composition. Examples of addable food comprising aforementioned extract therein are various food, beverage, gum, vitamin complex, health improving food and the like.

The present invention is more specifically explained by the following examples. However, it should be understood that the present invention is not limited to these examples in any manner.

### DESCRIPTION OF DRAWINGS

FIGS. 1 to 2 show the inhition effect of the hot-water extract of cucurbitaceae plant on the adipocytes(3T3-1) differentiation and the triglyceride level: FIG. 1 is a ph-tomicrograph of the accumulated fat within differentiated cell stained with Oil Red O staining reagent, FIG. 2 represents the inhibition percentage of the stained fat;

FIGS. 3 to 4 show the inhibition effect of each solvent soluble fractions of the stem of pumpkin on the adipocytes(3T3-1) differentiation and the triglyceride level; FIG. 3 represents the accumulated fat within differentiated cell stained with Oil Red O staining reagent, FIG. 4 represents the inhibition percentage of the stained fat;

FIG. 5 represents the inhibition effect of cmc-9 purified extract of the stem of pumpkin on the adipocytes(3T3-1) differentiation and the triglyceride level;

FIG. 6 represents the regulating effect of the hot-water extract of the stem of pumpkin on the gene expression when the adipocytes differentiate;

FIG. 7 represents the effect of the hot-water extract, each solvent soluble fractions and cmc-9 purified extract of the stem of pumpkin on the PPARs activity;

FIGS. 8 to 9 represent the effect of the hot-water extract of the pumpkin(PG105) on the blood triglyceride concentration in the high-fat diet mice; FIG. 8 shows the result of the group treated samples after 6 weeks, FIG. 9 shows the result of the group treated samples after 13 weeks,

FIGS. 10 to 11 represent the effect of the hot-water extract of the pumpkin(PG105) on the bold cholesterol concentration in the high-fat diet mice; FIG. 10 shows the result of the group treated samples after 6 weeks, FIG. 11 shows the result of the group treated samples after 13 weeks,

FIG. 12 shows the effect of the hot-water extract of the pumpkin(PG105) on the fatty liver in the high-fat diet mice for 13 weeks,

FIG. 13 shows the effect of the hot-water extract of the pumpkin(PG105) on the triglyceride in liver organ in the high-fat diet mice,

FIGS. 14 to 15 show the effect of the hot-water extract of the pumpkin(PG105) on the gene expression involved in fat metabolism,

FIG. 16 shows the redwing effect of the hot-water extract of the pumpkin(PG105) on the body weight in the obesity mouse model,

FIG. 17 shows the regulating effect of the hot-water extract of the pumpkin(PG105) on the obesity mouse model,

FIG. 18 shows the TLC (developing solvent; chloroform:methanol=20:1) result of the chloroform soluble fraction of cucurbitaceae plant,

FIG. 19 shows HPLC chromatogram of the purified extract(cmc-9) from the cucurbitaceae plant.

### MODE FOR INVENTION

It will be apparent to those skilled in the art that various modifications and variations can be made in the compositions, use and preparations of the present invention without departing from the spirit or scope of the invention.

The present invention is more specifically explained by the following examples. However, it should be understood that the present invention is not limited to these examples in any manner.

### EXAMPLES

The following Examples and Experimental Examples are intended to further illustrate the present invention without limiting its scope.

### Example 1

### Preparation of the Hot-Water Extract of Cucurbitaceae Plant

Each 2 kg of dried stems of pumpkin (Cucurbita moschata DUCH), water melon (Citrullus vulgaris SCHRAD), and sponge gourd (Luffa cylindrical L. ROEM) purchased from Korean market was cut, mixed with 201 of methanol and extracted for 1 hour at 90° C. with reflux extraction apparatus. Above extraction steps were repeated three times to collect supernatant, filtrated with filter paper and the supernatant was concentrated under reduced pressure to obtain 300 g, 350 g and 240 g of dried hot-water extract of pumpkin, water melon, and sponge gourd respectively. Each resulting extract was used in following Experimental Examples as test samples by dissolving in dist. water to the concentration of 100 mg/ml.

### Example 2

### Preparation of Hexane Soluble Extract

200 g of dried hot-water soluble extract of pumpkin prepared in Example 1 was suspended with 1 L of distilled water and 1 L of hexane was added thereto. The solution was subjected to fractionation with hexane layer and water layer three times and then the collected hexane layer was filtrated and dried with rotary vacuum evaporator to obtain 180 mg of hexane soluble extract. 1 L of remaining water layer was used in following steps.

### Example 3

### Preparation of Chloroform Soluble Extract

1 L of chloroform was added to 1 L of remaining water layer and the solution was subjected to fractionation with chloroform layer and water layer three times. The collected chloroform layer was filtrated and dried with rotary vacuum evaporator to obtain 770 mg of chloroform soluble extract. Remaining water layer was used in following steps.

### Example 4

### Preparation of Ethylacetate Soluble Extract

1 L of ethylacetate was added to 1 L of remaining water layer and the solution was subjected to fractionation with ethylacetate layer and water layer three times. The collected ethylacetate layer was filtrated and dried with rotary vacuum evaporator to obtain 2.1 g of ethylacetate soluble extract. Remaining water layer was used in following steps.

### Example 5

### Preparation of Butanol Soluble Extract

1 L of butanol was added to 1 L of remaining water layer and the solution was subjected to fractionation with butanol layer and water layer three times. The collected butanol layer was filtrated and dried with rotary vacuum evaporator to obtain 7.4 g of butanol soluble extract. Remaining water layer was used in following steps.

### Example 6

### Preparation of Purified cmc-9 Extract

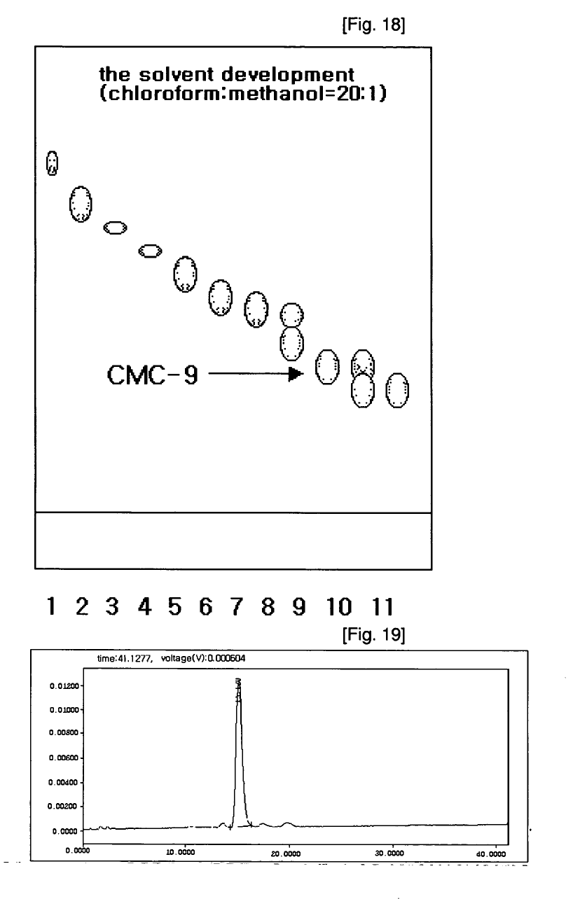

770 mg of chloroform soluble extract was subjected to Silica gel column chromatography using column (3×27 cm) filled with 25 g of Silica gel (Merck Co. No-9385) and eluting with solvent mixture (hexane: chloroform:methanol=16:15;1) as a mobile phase. Collected fractions were dried to afford 11 fractions, i.e., 1^{st} fraction (31 mg), 2^{nd} fraction (18 mg), 3^{rd} fraction (65 mg), 4^{th} fraction (18 mg), 5^{th} fraction (54 mg), 6^{th} fraction (75 mg), 7^{th} fraction (39 mg), 8^{th} fraction (200 mg), 9^{th} fraction (20 mg), 10^{th} fraction (163 mg) and 11^{th} fraction (64 mg). 20 mg of 9^{th} fraction showing most potent anti-obesity activity was subjected to silica gel column chromatography filled with 2 g of silica gel (Merck 9385) and eluted with a stepwise application of solvent mixture containing linear gradient of chloroform:methanol (30:1 ?>10:1). To purify the 9th fraction further, HPLC using methanol ranging from 20 to 70% as a mobile phase and running 40% methanol with a flow velocity of 2 ml/m was performed to obtain inventive ‘cmc-9’ extract eluted at 26.8 min. The cmc-9 extract was eluted at 15 min which shows 0.32 of R value in TLC eluting solvent system (chloroform:methanol=20:1) as can be seen in FIGS. 18 and 19.

### Experimental Example 1

### Inhibition Effect on the Adipocyte Differentiation and Triglyceride Level

To determine the inhibiting activity of the extract prepared by Examples 1 to 6 on the differentiation and triglyceride level, following experiment was performed.

1-1. The Crude Extract of Test Samples

Adipocyte cell (3T3-L1) purchased from ATCC (American Tissue Culture Collection, USA) was cultured in RPMI medium containing 10% FBS and MDI cocktail (isobutylmethylxanthine, dexamethasone, insulin) was added thereto for differentiating into mature adipocytes cell. After two days, the medium was replaced and treated with only insulin. Thereafter, the medium was replaced and equal concentration of insulin was treated again every other day. MDI ranging from 2.5 to 1000 ug/ml of the concentration was treated when the adipocytes differentiation was induced and equal concentration of the MDI was treated at every replacement of the medium.

Troglitazone (Sigma Co.) and 10 uM SB203580 (Sigma Co.) were treated as control groups and each 1 mg/ml of dried hot-water extract of pumpkin, water melon, and sponge gourd was treated thereto as respective test sample group. After eight days lapsed, the accumulated fat within differentiated cell was stained with Oil Red O staining reagent and the absorbance was determined with optical density qualitatively. The inhibition percentage (%) was calculated by following Empirical formula 1.
The Inhibition Percentage (%)=[O.D. of test group]/[O.D. value of Control group]×100 [Empirical Formula 1]

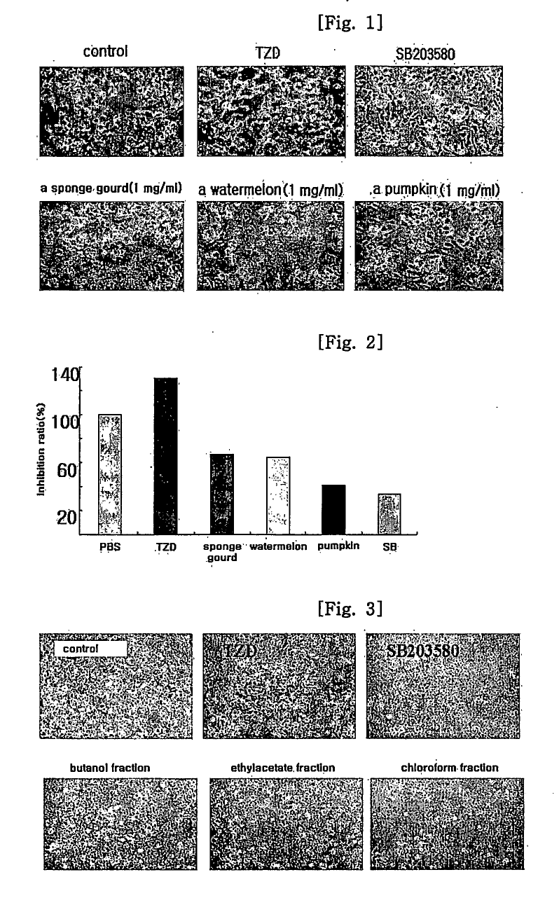

As the result, it is confirmed that precursor fat cell, 3T3-L1 cell was differentiated into mature adipocytes and triglyceride was accumulated in the MDI treatment group. In the troglitazone treatment group, the triglyceride was more produced and the red color was more intensified than those of other groups while the triglyceride was not produced in the SB203580 treatment group. The level of produced triglycerides in test sample treatment group, i.e., each extract of pumpkin, water-melon, and sponge gourd treatment group, especially pumpkin treatment group was significantly reduced in a dose dependent manner (See FIGS. 1 and 2).

1-2. Each Solvent Soluble Fractons of Test Samples

To determine the inhibiting activity of the chloroform, ethylacetate and butanol soluble fractions isolated from the stem of pumpkin prepared by Example 3 to 5 on the adipocyte differentiation and triglyceride level, identical experiment to the above-described steps disclosed in Expeimental Example 1-1 was performed wherein the treated concentration of each fraction was 100 ug/ml.

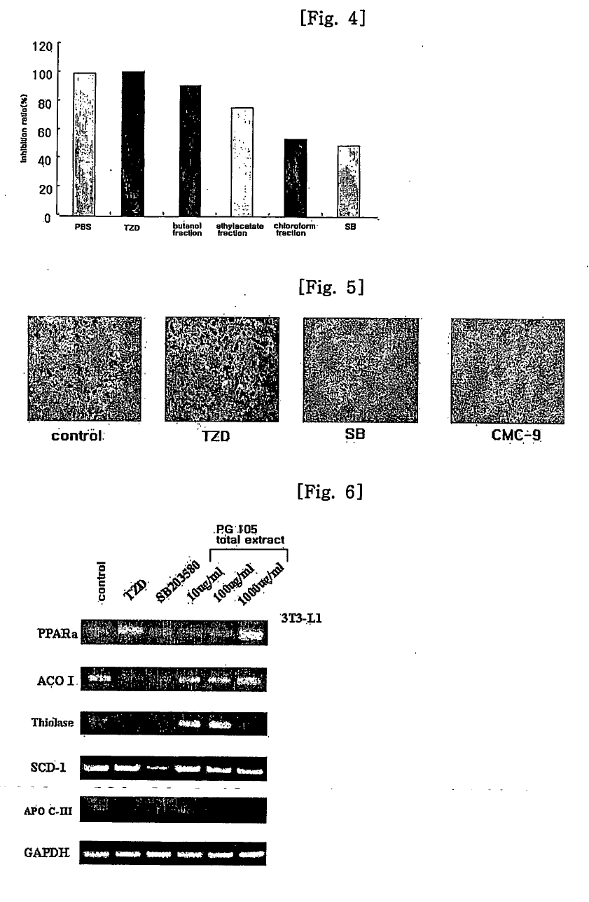

As the result, it is confirmed that the chloroform and ethylacetate soluble fractions showed potent inhibiting activity of the adipocyte differentiation and the production of triglyceride and especially, the chloroform soluble fraction showed most potent inhibition effect on adipocyte differentiation (See FIGS. 3 and 4).

1-3. “cmc-9” Purified Extract of Test Sample

To determine the inhibiting activity of the cmc-9 purified extract isolated from the stem of pumpkin prepared by Example 6 on the adipocyte differentiation and triglyceride level, identical experiment to the above-described steps disclosed in Experimental Example 1-1 was performed.

As can be seen in FIG. 5, it is confirmed that the triglyceride was more reproduced and the red color was more intensified in the troglitazone treatment group than those of other groups while the triglyceride was not formed in the SB203580 treatment group.

The cmc-9 treatment group showed most potent inhibiting effect on adipocyte differentiation and triglyceride accumulation.

### Experimental Example 2

### Regulation Effect on the Gene Expression in the Adipocyte Differentiation Process

To determine the regulating activity of the extract prepared by Examples 1 to 6 on the gene expression in the adipocyte differentiation process, following experiment was performed.

At first, the differentiation of precursor fat cell 3T3-L1 was induced by MDI treatment. Troglitazone (Sigma Co. USA), a PPAR gamma activator and SB 203508 (Sigma Co. USA), a p38 inhibitor inhibiting cell division were treated as control groups. Various concentrations i.e., 1 mg/ml, 10 mg/ml, and 100 mg/ml of hot water extract of pumpkin prepared by Example 1 (called as PG-105 hereinafter), was treated therewith. Each treated cell was incubated at 37° C. for 10 days replacing its medium. The cultured cells were washed with cold saline solution twice. The RNA thereof was extracted by TRiAzol agent, subjected to reverse transcription by reverse transcriptase enzyme to obtain its cDNA and all the factors, i.e., PPAR alpha, ACOI, Thiolase, Apo C-III, SCD-I, GAPDH etc were amplified. The difference between amplified gene expressions was compared by using agarose gel electrophoresis and the primer sequence used in each gene amplification was explained in Sequence Lists 1 to 12.

As can be seen in FIG. 6, the cell treated with 1 mg/ml of PG 105 showed increased expression of PPAR alpha, ACOI and Thiolase reported to be activated by PPAR alpha, whereas Apo C-III expression was reduced.

### Experimental Example 3

### Regulation Effect on the PPAR Alpha Activation

To determine the regulating activity of the extract prepared by Examples 1 to 6 on the PPAR alpha activation, following experiment was performed.

The hexane, chloroform, and ethylacetate soluble fractions prepared by Examples 2 to 4 and cmc-9 purified extract prepared by Example 6 were used as test samples in following experiment.

Two kind of transformed CV-1 cell group, i.e. the transformed group with only tkPPRE luciferase reporter plasmid and the transformed group with both of reporter plasmid and one vector expressing PPAR alpha, delta or gamma simultaneously were prepared and 24 hours later, test samples including PG 105 and other fractions were treated thereto to recover the cells after 24 hours and determine their luciferase activity. As control drugs, 100 uM fenofibrate (F6020-100G, Sigma Co.) was treated thereto in case of PPAR alpha, 10 uM GW501516 (Sigma Co.) in case of PPAR delta and 100 uM Troglitazone in case of PPAR gamma.

As can be seen in FIG. 7, the group treated with only tkPPRE showed no luciferase activity while the group treated with PPAR alpha simultaneously showed potent luciferase activity in the groups treated with chloroform and hexane soluble fractions, of which activity is about 60 fold higher as compared with that in negative control group treated with only ethanol and more than five fold higher as compared with that in positive control group treated with fenofibrate (Sigma Co.). In case that PPAR delta was transformed, the luciferase activity in the group treated with hexane fraction showed about 20 fold higher as compared with that in negative control and similar to that in positive control group treated with GW501516. However, in case that PPAR gamma was transformed, the test samples treated with PG 105, various fractions and cmc-9 extract showed no activity.

Accordingly, it is confirmed that the extract of Cucurbitaceae family plant can regulate lipid metabolism.

### Experimental Example 4

### Inhibition Effect on the Accumulation of Triglyceride in High-Fat Diet Mouse

To determine the inhibiting activity of the extract prepared by Examples 1 to 6 on the accumulation of triglyceride in high-fat diet mouse, following experiment was performed.

4-1. Pretreatment

8 to 10 weeks old C57/BL mice weighing from 18 to 20 g (Jackson Lab, USA) were put into ventilating box and classified into five groups. Normal feed was supplied to group 1 and high-fat feed containing more than 45% fat (Jackson Lab. USA) was supplied to remaining four groups, i.e., groups 2 to 5. Distilled water was administrated into groups 1 and 2, and 8 mg of PG105 prepared by Example 1 was administrated into group 3 everyday for 16 weeks orally. 40 ug of Xenical (Roche Co.) was administrated into group 4 and 5 ug of Reductil (Abbot Co.) was orally administrated into group 5 everyday. Respective 6 weeks and 13 weeks after the administration, each blood sample was collected and their blood triglyceride level and cholesterol level were determined. 13 weeks later, the mice were killed to death and their livers were isolated to determine their blood triglyceride level. mRNA was isolated from fat organ and liver, and the gene expression concerning with lipid absorption and metabolic pathway was investigated using by RT-PCR method.

4-2. The Effect on the Blood Triglyceride and Cholesterol

Respective 6 weeks and 13 weeks after the administration, each blood sample was collected and blood triglyceride level and cholesterol level were determined.

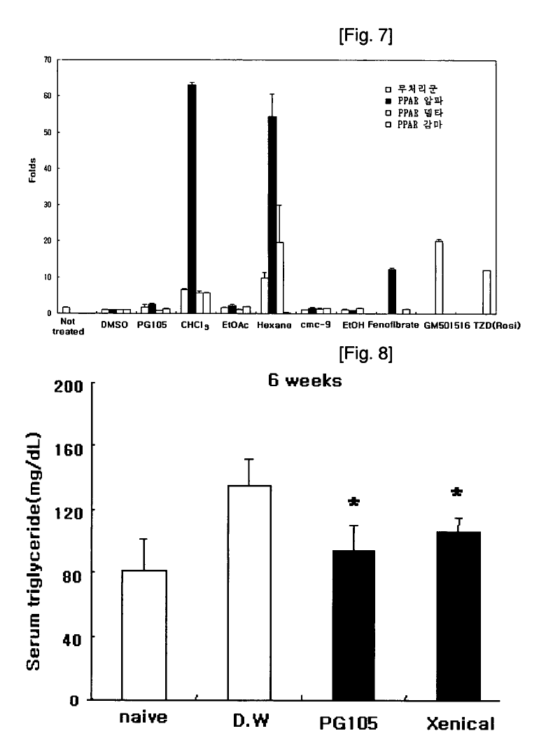

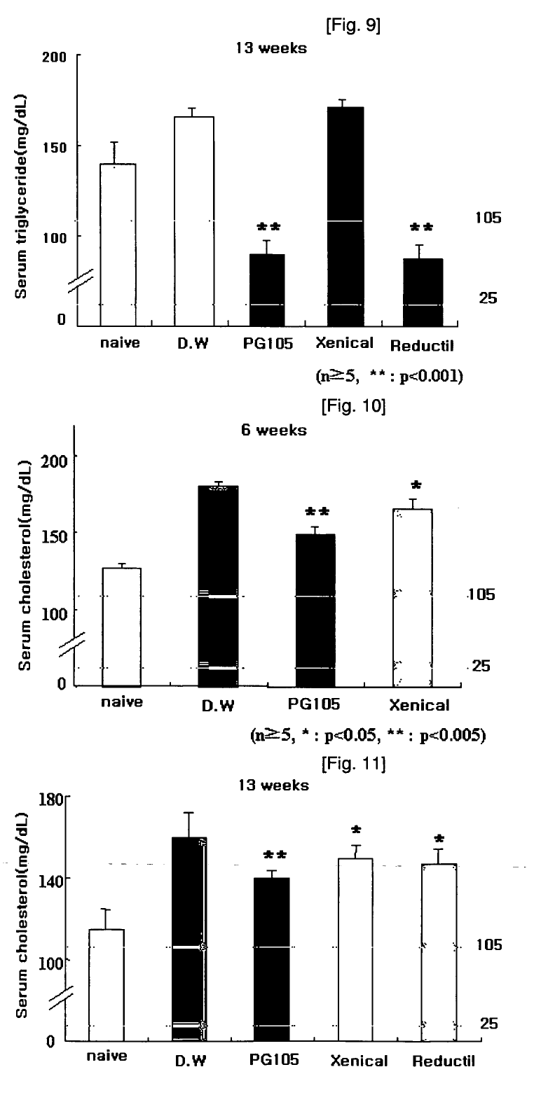

At the result, the group 2, high-fat diet group treated with only distilled water showed 134.67 mg/dl and 169.33 mg/dl of blood triglyceride concentration after 6 weeks and 13 weeks respectively, which showed 1.66 fold higher as compared with that of no high-fat diet groups. Contrary to the group, the group 3 treated with PG 105 showed 94 mg/dl and 90 mg/dl of blood concentration after 6 weeks and 13 weeks respectively, which showed similar to that of no high-fat diet groups. (See FIGS. 8 and 9).

The group 2 treated with only distilled water showed 180 mg/dl while the group 3 treated with PG 105 showed 148.6 mg/dl of blood cholesterol concentration (a FIGS. 10 and 11).

4-3. The Effect on the Fatty Liver

At the result of observing their livers by naked eye, the high-fat diet group treated with only distilled water showed yellowish fat liver while the high-fat diet group treated with PG 105 extract showed brightly red colored liver at 13 weeks after the administration (See FIG. 12).

4-4. The Effect on the Accumulation of Triglyceride in Liver Organ

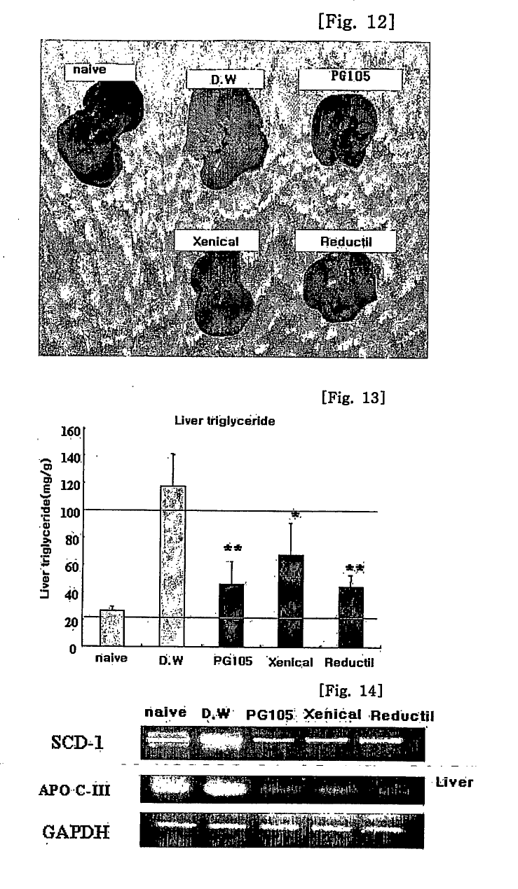

The level of accumulated triglyceride in liver organ at 13 weeks after the administration was determined. At the result, the high-fat diet mice treated with distilled water showed 117.53 mg/dl of accumulated triglyceride level, which showed five fold higher as compared with that of no high-fat diet groups and excessive accumulation of triglyceride in liver organ. Contrary to the result, the group treated with PG 105 showed 46.15 mg/dl of triglyceride concentration, of which value is within the range of normal level and is consistent with the observed result with naked eye. (See FIG. 13).

4-5. The Effect on the Gene Expression Involved in Fat Metabolism

To investigate the effect of the extract of Cucurbitaceae plant on the gene expression involved in fat metabolism including lipolysis and lipogenesis, RNA was isolated from adipocyte and liver organ and subjected to RT-PCR method to investigate the change of gene expression involved in fat metabolism.

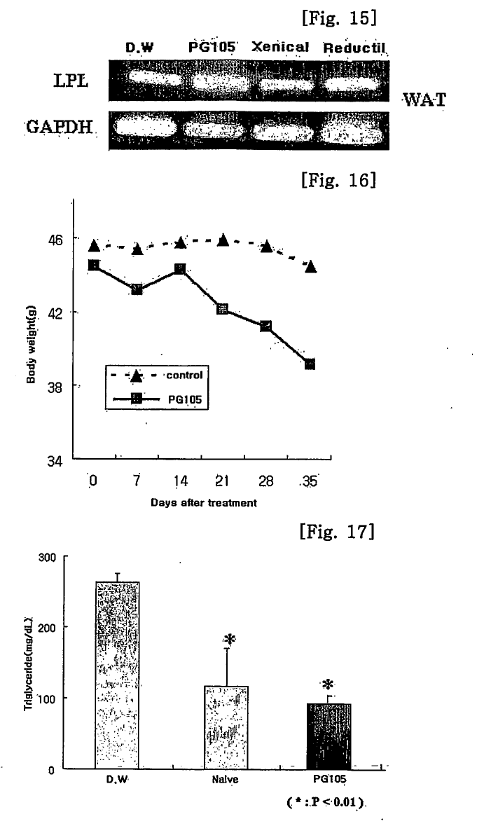

At the result, the expression of lipoprotein lipase (LPL), an enzyme involved in the dissociation of triglyceride accumulated in adipocyte organ into fatty acid disclosed in the literature (Schonfeld, G., et al., Metab. Cin. Exp., 28, pp1001-1009, 1979) was significantly increased by the administration of PG105 and the expression of APO CIII protein reported to inhibit the action of lipoprotein lipase enzyme (Windler E. et al., J. Biol. Chem., 255, pp8303-8307, 1980; Wang C. S. et al., J. Cin. Invest., 75 pp384-390, 1985) was significantly inhibited (See FIGS. 14 and 15).

As can be seen in FIG. 10a, the expression of SCD (Stearoyl-CoA desaturase) was significantly decreased. The SCD is reported to be an enzyme introducing cis double bond into between 9 and 10 position of palmitoyl-CoA and stearoyl-CoA as a substrate to produce palmitoeoyl-CoA and oleoyl-CoA (Enoch, H. G. et al., J. Biol. Chem., 251, pp5095-5103, 1976), which is further used in constituting phospholipid, cholesterol and triglyceride etc (Ntambi, J. M., J. Lipid Res., 40, pp1549-1558, 1999).

Those results suggested that the oral administration of PG 105 showed dual actions, i.e., promoting activity of lipolysis as well as inhibiting activity of lipid synthesis.

### Experimental Example 5

### Inhibition Effect on the Accumulation of Triglyceride in High-Fat Diet Mouse

To determine the reducing effect on the body weight and regulating effect on the lipid level of the extract prepared by Examples 1 to 6 in obesity mouse model (db/db), following experiment was performed.

5 weeks old female obesity mice (Jackson Lab, USA) were bred allowing freely access to water and fed up to 8 weeks and 8 mg of PG 105 for each mouse was orally administrated into each mouse for 6 weeks by dissolving in 100 ml of distilled water. Sterilized distilled water was administrated to the mice as a negative control. The change of bodyweight was determined once a week and the amount of feed intake was determined everyday. The blood lipid level of the mice pooled at 3^{rd} and 6^{th} week was also determined.

At the result, the group treated with PG 105 showed significant reducing effect on the body weight of mice to the extent that the body weight was reduced by about 10 to 15% at 6^{th} week (See FIG. 16).

The group treated with PG 105 showed significant decreasing effect on the blood triglyceride level of mice to the extent that the blood triglyceride level was 150 mg/dl at 6^{th} week (Se FIG. 17).

### Experimental Example 6

### Reducing Effect on the Body Weight and Regulating Effect on the Lipid Level in Human Volunteers

To determine the reducing effect on the body weight and regulating effect on the lipid level of the extract prepared by Examples 1 to 6 in human volunteers, following clinical experiment was performed.

The hot-water extract extracted from the stem of sponge gourd was orally administrated to six volunteers consisting of four women aged from 27 to 62 years old and two men aged from 30 to 48 years old in the dose ranging from 2 to 5 g/days for two weeks and the body weight (kg) and the size of waist girth (cm) of the volunteers were determined. The total lipid level (mg/dl) was determined by diagnosis kit (total lipid reagent, Vediees Co. USA) using colorimetric method (photometer, Agilent 8453, Agilent Co. USA) and the FFA level (Free fatty acid, uEq/l) was determined by Hitari (Hitachi 7150, Hitachi Co. Japan) using by enzymatic method (Sicdia NEFAYME, EKEN Co. Japan). The cholesterol level (mg/dl) was determined by ADVIA (ADVIA 1655, Bayer Co. Japan) using by cholesterol kit (cholesterol reagent, Bayer Co. USA) and the VLDL-cholesterol level (mg/dl) was determined by Spectrophotometer (Photometer 4020, Roche Co. Germany) using by diagnosis kit (BLF II, EKEN Co., Japan). The LDL-cholesterol level (mg/dl) was determined by Hitacri (7153, Hitachi Co. Japan) using by LDL-cholesterol kit (LDL-cholesterol reagent, Roche Co. Germany) and the HDL-cholesterol level (mg/dl) was determined by ADVIA (ADVIA 1650, Bayer Co. Japan) using by diagnosis kit (Direct HDL-Cholesterol, Bayer Co. UK). The triglyceride level (mg/dl) was determined by ADVIA (ADVIA 1650, Bayer Co. Japan) using by triglyceride kit (triglyceride reagents, Bayer Co. USA) and the glucse level (mg/dl) was determined by ADVIA (ADVIA 1850, Bayer Co. Japan) using by diagnosis kit (Glucose Hexokinase, Bayer Co., USA). The SGOT and SGPT (U/I) levels were determined by ADIVIA (ADIVIA 1655, Bayer Co. Japan) using by AST reagent kit (Bayer Co. USA) and ALT reagent kit (Bayer Co. USA).

At the result, the hot water extract extracted from the stem of sponge gourd showed potent redwing effect on the body weight and regulating effect on the lipid metabolism as can be seen in following Table 1.

TABLE 1

| Content | At the beginning | 2^{nd} weeks | Normal level |
|---|---|---|---|
| Body weight (Kg) | 67.7 ± 10.9 | 66.9 ± 10.3 | |
| Size of waist girth | 84.9 ± 7.2 | 84.8 ± 9.6 | |
| (cm) | | | |
| Total lipid (mg/dl) | 668.0 ± 126.6 | 652.8 ± 192.7 | 400-1000 |
| FFA (uEq/l) | 602.3 ± 240 | 232.2 ± 147.5 | 170-585 |
| Cholesterol (mg/dl) | 231.5 ± 42.4 | 208.0 ± 39.9 | <220 |
| VLDL-Cholesterol | 242.0 ± 94.9 | 225.6 ± 96.2 | 75-200 |
| (mg/dl) | | | |
| LDL-Cholesterol | 139.2 ± 31.9 | 123.4 ± 26.2 | <130 |
| (mg/dl) | | | |
| HDL-Cholesterol | 59.8 ± 7.3 | 55.8 ± 7.2 | 35-80 |
| (mg/dl) | | | |
| Triglyceride | 171.3 ± 73.1 | 114.6 ± 96.7 | <200 |
| (mg/dl) | | | |
| Glucose (mg/dl) | 106.8 ± 27.5 | 116.4 ± 75.4 | 70-110 |
| SGOT (U/l) | 25.1 ± 8.1 | 21.3 ± 1.5 | 31(F) 37(M) |
| SGPT (U/l) | 25.6 ± 12.5 | 22.8 ± 11.3 | 31(F) 40(M) |

Accordingly, it is confirmed that not only the stem extract of pumpkin but also the stem extract of sponge gourd belong to identical family showed potent anti-obesity activity through above described clinical example.

### Experimental Example 7

### Toxicity test

Methods

The acute toxicity tests for the test samples on six weeks aged SPF SD rats were performed by following procedure.

Four groups consisting of 2 rats was administrated orally with 100 mg/kg of the PG 105 extract and observed for 2 weeks.

Results

There were no treatment-related effects on mortality, clinical signs, body weight changes and gross findings in any group or either gender. The minimum LD₅₀ value in oral administration was more than 100 g.kg. These results suggested that the test compounds prepared in the present invention were potent with safe.

Hereinafter, the formulating methods and kinds of excipients will be described, but the present invention is not limited to them. The representative preparation examples were described as follows.

Preparation of Powder

| | |
|---|---|
| PG 105 | 50 mg |
| Lactose | 100 mg |
| Talc | 10 mg |

Powder preparation was prepared by mixing above components and filling sealed package.

Preparation of Tablet

| | |
|---|---|
| PG 105 | 50 mg |
| Corn Starch | 100 mg |
| Lactose | 100 mg |
| Magnesium Stearate | 2 mg |

Tablet preparation was prepared by mixing above components and entabletting.

Preparation of Capsule

| | |
|---|---|
| PG 105 | 50 mg |
| Corn starch | 100 mg |
| Lactose | 100 mg |
| Magnesium Stearate | 2 mg |

Tablet preparation was prepared by mixing above components and filling gelatin capsule by conventional gelatin preparation method.

Preparation of Injection

| | |
|---|---|
| cmc-9 extract | 50 mg |
| Distilled water for injection | optimum amount |
| PH controller | optimum amount |

Injection preparation was prepared by dissolving active component, controlling pH to about 7.5 and then filling all the components in 2 mg ample and sterilizing by conventional injection preparation method.

Preparation of Liquid

| cmc-9 extract | 0.1˜80 g |
|---|---|
| Sugar | 5˜10 g |
| Citric acid | 0.05˜0.3% |
| Caramel | 0.005˜0.02% |
| Vitamin C | 0.1˜1% |
| Distilled water | 79˜94% |
| CO₂ gas | 0.5˜0.82% |

Liquid preparation was prepared by dissolving active component, filling all the components and sterilizing by conventional liquid preparation method.

Preparation of Health Care Food

| cmc-9 extract | 1000 | mg |
|---|---|---|
| Vitamin mixture | optimum | |
| | amount | |
| Vitamin A acetate | 70 | mg |
| Vitamin E | 1.0 | mg |
| Vitamin B₁ | 0.13 | mg |
| Vitamin B₂ | 0.15 | mg |
| Vitamin B₆ | 0.5 | mg |
| Vitamin B₁₂ | 0.2 | mg |
| Vitamin C | 10 | mg |
| Biotin | 10 | mg |
| Amide nicotinic acid | 1.7 | mg |
| Folic acid | 50 | mg |
| Calcium pantothenic acid | 0.5 | mg |
| Mineral mixture | optimum | |
| | amount | |
| Ferrous sulfate | 1.75 | mg |
| Zinc oxide | 0.82 | mg |
| Magnesium carbonate | 25.3 | mg |
| Monopotassium phosphate | 15 | mg |
| Dicalcium phosphate | 55 | mg |
| Potassium citrate | 90 | mg |
| Calcium carbonate | 100 | mg |
| Magnesium chloride | 24.8 | mg |

The above-mentioned vitamin and mineral mixture may be varied in many ways. Such variations are not to be regarded as a departure from the spirit and scope of the present invention.

Preparation of Health Beverage

| | | |
|---|---|---|
| PG105 | 1000 | mg |
| Citric acid | 1000 | mg |
| Oligosaccharide | 100 | g |
| Apricot concentration | 2 | g |
| Taurine | 1 | g |
| Distilled water | 900 | ml |

Health beverage preparation was prepared by dissolving active component, mixing, stirred at 85° C. for 1 hour, filtered and then filling all the components in 1000 ml ample and sterilizing by conventional health beverage preparation method.

The invention being thus described, it will be obvious that the same may be varied in many ways. Such variations are not to be regarded as a departure from the spirit and scope of the present invention, and all such modifications as would be obvious to one skilled in the art are intended to be included within the scope of the following claims.

### INDUSTRIAL APPLICABILITY

As described in the present invention, the extract of Cucurbitaceae family plant of the present invention showed potent reducing activity of body weight, decreasing effect on the blood triglyceride and cholesterol level, activating activity of PPAR alpha and delta, reducing activity of the gene expression of stearoyl-CoA desaturase, and preventing activity from the adipogenesis of precursor fat cells with no toxicity, therefore, those extract can be useful in treating or preventing obesity and adipogenesis-involved diseases as a medicine or health care food.

1. A purified extract isolated from Cucurbitaceae family plant prepared by the steps consisting of:
adding about 5 to 15 fold volume of distilled water to dried material of Curcubitaceae family plant,
extracting the plant to obtain plant extract,
filtrating and drying the extract with reduced pressure to obtain hot water-soluble extract of the plant at 1st step;
suspending said hot water-soluble extract with water and subjecting fractionation with hexane, chloroform, ethylacetate, butanol solvent with increasing order of polarity to obtain respective organic solvent-soluble fraction at 2nd step;
subjecting said chloroform-soluble fraction to silica gel column chromatography with a solvent mixture mixed with hexane: chloroform:methanol (16:15:1) to afford 11 sub-fractions at 3rd step;
subjecting 9th fraction among said sub-fractions to repetitive silica gel column chromatography with a solvent mixture mixed with chloroform:methanol and HPLC to obtain inventive purified extract designated to “cmc-9” having potent anti-obesity and anti-adipogenic activity and showing TLC spectrum as in FIG. 12

2. A pharmaceutical composition comprising the extract of Cucurbitaceae family plant or the purified extract as set forth in claim 1 having anti-adipogenic and anti-obesity activity as an active ingredient in an effective amount to treat and prevent obesity and adipogenesis-involved diseases.

3. The pharmaceutical composition of claim 2, wherein said Cucurbitaceae family plant is selected from the group consisting of pumpkin (Cucurbita moschata DUCH), water-melon (Citrullus vulgaris SCHRAD), sponge gourd (Luffa cylindrical L. ROEM), gourd (Lagenaria siceraria STANDL. var. depressa HERA), and cucumber (Cucumis sativus L).

4. The pharmaceutical composition of claim 3, wherein said Cucurbitaceae family plant is selected from pumpkin (Cucurbita moschata DUCH), water melon (Citrullus vulgaris SCHRAD), and sponge gourd (Luffa cylindrical L. ROEM).

5. The pharmaceutical composition of claim 2, wherein said extract of Cucurbitaceae family plant is extracted from the stem or leaf thereof.

6. The pharmaceutical composition of claim 2, wherein said obesity and adipogenesis-involved diseases is selected from the group consisting of obesity, type II diabetes, steatosis, hyperlipemia, cardiovascular disease, and artherosclerosis.

7. The pharmaceutical composition of claim 2, wherein said extract is selected from the group consisting of crude extract and non-polar solvent soluble extract.

8. The pharmaceutical composition of claim 2, wherein said extract is hot-water soluble extract.

9. The pharmaceutical composition of claim 7, wherein said non-polar solvent is selected from the group consisting of hexane, chloroform, dichloromethane, and ethylacetate.

10. The pharmaceutical composition of claim 9, wherein said non-polar solvent is chloroform.

11. The pharmaceutical composition claim 2, wherein the pharmaceutical composition contains about 0.02˜90% by weight of the extract based on the total weight of the pharmaceutical composition.

12. health care food comprising a crude extract, non-polar solvent soluble extract of Cucurbitaceae family plant or cmc-9 extract as set forth in claim 1, together with a sitologically acceptable additive for the prevention and alleviation of obesity and adipogenesis-involved diseases.

13. The health care food of claim 12, wherein said food is heath care beverage.


SEQUENCE LISTING (12 sequences)

SEQ ID NO: 1 - DNA, 20 bp, Artificial Sequence
  PPARalpha primer 1
gtgacagacaacggcagtcc

SEQ ID NO: 2 - DNA, 20 bp, Artificial Sequence
  PPARalpha primer2
atgttcagttccacaccggg

SEQ ID NO: 3 - DNA, 21 bp, Artificial Sequence
  ACO 1 primer1
ctcactcgaagccagcgttac

SEQ ID NO: 4 - DNA, 16 bp, Artificial Sequence
  ACO 1 primer2
gtctgcctctacccgg

SEQ ID NO: 5 - DNA, 21 bp, Artificial Sequence
  Thiolase primer2
cgctagttacttagtgcatac

SEQ ID NO: 6 - DNA, 16 bp, Artificial Sequence
  Thiolase primer2
ggattgtcgactcggt

SEQ ID NO: 7 - DNA, 21 bp, Artificial Sequence
  Apo C-111 primer1
gggctctgtgcagggctacat

SEQ ID NO: 8 - DNA, 16 bp, Artificial Sequence
  Apo C-111 primer2
ttcaagtggccgaaga

SEQ ID NO: 9 - DNA, 25 bp, Artificial Sequence
  SCD-1 primer1
acatgtctgacctgaaagccgagaa

SEQ ID NO: 10 - DNA, 25 bp, Artificial Sequence
  SCD-1 primer2
cctctgccctcagtgttctcatcga

SEQ ID NO: 11 - DNA, 19 bp, Artificial Sequence
  GAPDH primer1
gccttccgtgttcctaccc

SEQ ID NO: 12 - DNA, 21 bp, Artificial Sequence
  GAPDH primer2
ccatactgttacttatgccga